(19) United States
(12) Patent Application Publication
(10) Pub. No.: US 2024/0238361 A1
(43) Pub. Date: Jul. 18, 2024

(54) METHODS OF TREATING FIBROSIS AND HYPOXIA-ASSOCIATED DAMAGE TO PERIPHERAL NERVES

(71) Applicant: Temple Therapeutics BV, Geleen (NL)

(72) Inventors: Nilabh Ghosh, Basel (CH); Raphael Guzman, Allschwil (CH)

(73) Assignee: Temple Therapeutics BV, Geleen (NL)

(21) Appl. No.: 18/559,243

(22) PCT Filed: May 4, 2022
(86) PCT No.: PCT/IB2022/054134

§ 371 Date: Nov. 6, 2023

Related U.S. Application Data
(60) Provisional application No. 63/185,456, filed on May 7, 2021.

(51) Int. Cl. A61K 38/05; A61K 31/198; A61P 25/02

(52) U.S. Cl. CPC A61K 38/05; A61K 31/198; A61P 25/02

(57) ABSTRACT

Described herein are methods and compositions for treating and preventing peripheral nerve injury, and more particularly hypoxia-associated tissue damage such as peripheral nerve fibrosis. The methods described include the administration of a therapeutically effective amount of a source of glutamine to peripheral nerves or one or more tissues surrounding peripheral nerves.

### BACKGROUND

### 1. Technical Field text

The present invention relates to the treatment of hypoxia-associated tissue damage in subjects. More particularly, the methods relate to treating hypoxia-associated tissue damage in a subject during a surgical procedure according to particular dosing regimens.

### 2. BACKGROUND INFORMATION

The peripheral nerve system is a network of forty-three pairs of motor and sensory nerves that connect the brain and the spinal cord (the central nervous system) to the entire body. These nerves control the functions of and sensation, movement and motor coordination. The nerves are fragile and peripheral nerve injury (“PNI”) can be caused from traumatic injuries or diseases. Peripheral nerve fibrosis associated with chronic inflammation, perineural adhesions and scaring are often reported in patients with PNI.

The severity of PNI can be influenced through the presence of various pathophysiological conditions including hypoxia. Chronic hypoxia and duration of hypoxia has been reported to induce peripheral polyneuropathy. On a molecular level, responses to hypoxia are orchestrated by hypoxia-inducible factors (HIFs), consisting of an oxygen-dependent α-subunit (HIF-1α, HIF-2α) and an oxygen-independent β-subunit. HIF-α-subunits are constitutively expressed and rapidly degraded in normoxia. In hypoxia, however, HIF-1α and HIF-2α are stabilized and form active transcription complexes. The complexes bind to hypoxia response elements (HRE) in the promoter region of numerous downstream target genes, which collectively mount the adaptive response to hypoxia. Under hypoxic conditions the a subunit translocate to the nucleus and forms a heterodimer with the β subunit. The HIF heterodimer binds to the hypoxic responsive elements and further associates with other co-activators to regulate gene expression. Research over the last few years have shown that HIF1α signaling is involved in promoting fibrosis through the activation and production of excessive ECM. Excessive deposition of ECM factors such as collagen, fibronectin and proteoglycans are characteristic features of tissue fibrosis and HIF1α has been found to be involved in upregulation of ECM factors under hypoxic conditions. Further, an elevated expression of HIF1α in fibrotic diseases further indicates its involvement in pathogenesis of fibrosis.

### SUMMARY OF THE INVENTION

The present invention addresses the need for compounds and methods for treating and preventing peripheral nerve injury, and more particularly hypoxia-associated tissue damage such as peripheral nerve fibrosis. The methods include the administration of a therapeutically effective amount of a source of glutamine to peripheral nerves or one or more tissues surrounding peripheral nerves.

One aspect is a method of treating or preventing peripheral nerve fibrosis in a subject, the method comprising administering to one or more peripheral nerves or one or more tissues surrounding one or more peripheral nerves, wherein the source of glutamine is administered prior to, during or after a surgery involving the one or more peripheral nerves. In some embodiments, one or more additional doses of the glutamine source is administered to the subject at specified periods of time after the first dose, at fixed intervals of time after the first dose, and/or just prior to closure of all surgical incisions. In some embodiments, the source of glutamine is administered during a surgery involving the one or more peripheral nerves. In some embodiments, the source of glutamine is an oligopeptide comprising glutamine. In some embodiments, the oligopeptide is a dipeptide. In some embodiments, the dipeptide is L-alanyl-L-glutamine.

Another aspect is a method of treating or preventing adhesion in one or more peripherial nerves in a subject that is undergoing a surgical procedure, the method comprising administering to a peripheral nerve or tissue surrounding one or more peripheral nerves, wherein the source of glutamine is administered prior to, during or after a surgery involving the one or more peripheral nerves.

Another aspect is a method of treating or preventing hypoxia-associated damage to one or more peripheral nerves of a subject, the method comprising administering to the subject during the surgical procedure a therapeutically effective amount of a glutamine source to a tissue subject to hypoxia-associated tissue damage, wherein the source of glutamine is administered prior to, during or after a surgery involving the one or more peripheral nerves.

Another aspect is a method of prophylactically reducing the incidence of fibrosis to one or more peripheral nerves of a subject comprising administering to the one or more peripheral nerves or one or more tissues surrounding the peripheral nerves an effective amount of glutamine.

Another aspect is a method for reducing HIF-1α in hypoxia-associated damaged peripheral nerves, the method comprising administering to one or more peripheral nerves or one or more tissues surrounding one or more peripheral nerves, wherein the source of glutamine is administered prior to, during or after a surgery involving the one or more peripheral nerves.

Another aspect is a kit for the treatment or reduction of peripheral nerve adhesion or fibrosis in a subject comprising an effective amount of a source of glutamine, wherein the source of glutamine is formulated as a dosage form selected from the group consisting of a surgical implantable film, an injectable, a topical cream and dosage form suitable for iontophoresis.

### BRIEF DESCRIPTION OF THE DRAWINGS

FIG. 1A is a schematic of primary rat fibroblasts isolation.

FIG. 1B illustrates morphology and phenotypic characteristics of isolated cells were identified and confirmed with ICC imaging for specific markers including α-SMA, HSP47 and Vimentin.

FIG. 1C illustrates primary fibroblasts used for experiments under three different experimental conditions following phenotypic characterization.

FIGS. 2A-2B shows results of cell proliferation and cell death of hypoxic primary rat fibroblasts treated with an embodiment of the present invention.

FIGS. 3A and 3B illustrates the reduction of expression of HIF-1α and pro-fibrotic factors in primary fibroblasts treated with an embodiment of the present invention.

FIGS. 4A-4E illustrates the reduction of expression of pro-fibrotic markers in an in-vitro chronic hypoxic model according to an embodiment of the present invention.

FIGS. 5A-5E illustrates the reduction of expression of pro-fibrotic markers in an in-vitro chronic hypoxic model according to an embodiment of the present invention.

FIGS. 6A and 6B illustrate the reduction of expression of FBN and other pro-fibrotic factors involved with peripheral nerve fibrosis according to an embodiment of the present invention.

FIGS. 7A-7C illustrate the reduction of expression of pro-fibrotic and adhesion biomarkers of hypoxic primary rat fibroblasts according to an embodiment of the present invention.

FIG. 8 illustrates the modulation of signaling pathways through the regulation of specific of pro-fibrotic and adhesion biomarkers of hypoxic primary rat fibroblasts according to an embodiment of the present invention.

FIGS. 9A and 9B illustrate the reduction of expression of pro-fibrotic markers in an in-vitro chronic hypoxic model according to an embodiment of the present invention.

FIGS. 10A and 10B illustrate the reduction of expression of pro-fibrotic markers in an in-vitro chronic hypoxic model according to an embodiment of the present invention.

### DETAILED DESCRIPTION OF THE DRAWINGS AND THE PRESENTLY

### Preferred Embodiments

The following paragraphs define in more detail the embodiments of the invention described herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein. All patents and publications cited herein are incorporated by reference herein in their entirety.

The term “glutamine source” or “source of glutamine” includes glutamine and its physiologically acceptable salts, as well as glutamine conjugates and peptides comprising glutamine as described further herein.

The terms “dosage” or “dose” or “dosage form” as used herein denote any form or formulation of the glutamine source that contains an amount sufficient to produce a therapeutic effect with a single administration or multiple administrations.

The term “surgical incision” as used herein means a wound made by a cutting instrument such as a scalpel, laser, or other cutting instrument prior to or during a surgical procedure, including incisions or points of entry made for laparoscopic or other minimally invasive surgical techniques.

The term “prevention of” as used herein includes the meaning of reducing. The amount of the reduction may be from about 0.001% to about 100%.

The term “effective amount” is an amount that achieves the desired effect. For example, in the present invention, the effective amount of one or more glutamine sources is an amount that, after one or more administrations, reduces or prevents hypoxia-associated tissue damage. In some embodiments, the effective amount is an effective amount of a source of glutamine that prevents or reduces adhesion in one or more tissues.

The term “drug load” as used herein refers to the wt % of the glutamine source relative to the total mass of the dosage form.

The terms “formulation” or “composition” as used herein refers to the glutamine source in combination with one or more pharmaceutically acceptable diluents and/or excipients.

The terms “extended release” or “sustained release” as used herein refers to a composition that releases an active ingredient according to a desired profile over an extended period under physiological conditions or in an in vitro test. By “extended period” it is meant a continuous period of time of at least about 1 hour; about 2 hours; about 4 hours; about 6 hours; about 8 hours; about 10 hours; about 12 hours; about 14 hours; about 16 hours; about 18 hours; about 20 hours about 24 hours; or even longer; specifically, over a period of about 18 hours under physiological conditions or in an in vitro assay.

The term “delayed release” as used herein refers to a composition that releases an active ingredient after a period of time, for example days, minutes, or hours, such that the active ingredient is not released initially. A delayed release composition may provide, for example, the release of a drug or active ingredient from a dosage form, after a certain period, under physiological conditions or in an in vitro test.

The term “treating” refers to administering a therapy in an amount, manner, or mode effective (e.g., a therapeutic effect) to improve a condition, symptom, disorder, or parameter associated with a disorder, or a likelihood thereof. In some embodiments, the term treating refers to the treatment or prevention of adhesion formation following surgery. In other embodiments, the term treating refers to the treatment or prevention of hypoxia-associated tissue damage in a patient undergoing a surgical procedure.

The term “prophylactically treating” refers to administering a therapy in an amount prior to incurring a condition, symptom, disorder, or parameter associated with a disorder, or reducing the likelihood thereof.

The term “subject” refers to any mammal, including animals and humans.

The subject may be a medical patient in need of treatment thereof. In one embodiment, the subject is a human.

The term “preventing” or “reducing” refers to preventing or reducing the progression of a disorder, such as fibrosis or adhesion, either to a statistically significant degree or to a degree detectable to one skilled in the art.

The term “substantially” as used herein means to a great or significant extent, but not completely.

As used herein, “a” or “an” means one or more unless otherwise specified.

Terms such as “include,” “including,” “contain,” “containing,” “having,” and the like mean “comprising.”

Described herein are methods for treating hypoxia-associated tissue damage and, in particular, preventing or treating peripheral nerve injuries and peripheral nerve fibrosis adhesion by administering a glutamine source at a particular time. The methods described herein comprise administering a glutamine source to a subject at least once within a specified period of time. In certain embodiments, the glutamine source is administered to the subject more than once. For example, the subject may receive a first dose of the glutamine source within a specified period of time followed by one or more additional doses of the glutamine source at specified periods of time, at fixed intervals of time, and/or just prior to closure of all surgical incisions, and/or for time periods after the completion of surgery.

Some embodiments described herein are methods of treating or reducing hypoxia-associated tissue damage or fibrosis to peripheral nerves. Fibrosis is characterized by excessive deposition of collagen, contraction of the ECM matrix, tissue damage and aberrant wound healing. Scarring and tissue fibrosis is driven by overactivation of effector cells including fibroblasts. Overactivation and differentiation of fibroblasts into myofibroblasts causes a differential abundance of collagen formation and formation of scar tissue. Excessive collagen deposition in peripheral nerves inhibits axonal regeneration at the injury site. Moreover, crosslinking of collagen induces tissue destruction which is resistant to protease degradation. Chronic injury to tissue fibrosis involves a series of biochemical signaling of several intra and extracellular factors. In addition to this, presence of hypoxic microenvironment can promote tissue damage and peripheral neuropathy.

HIF-1 acts on fibroblasts to regulate the ECM matrix under hypoxic conditions thereby making it a crucial regulator of the tissue architecture and fibrosis. Together, compelling evidence suggests HIF1α modulates the complexity of fibrosis of various organs and signaling of HIF1α is of important clinical relevance. Its role on the pathological outcome of fibroblasts and macrophages makes it a suitable target for pharmacological and genetic inhibition in PNI as well. Furthermore, several components of the HIF-1 signaling cascade regulates biochemical signaling of various extracellular growth factors further indicating its varied role in tissue remodeling.

Glutamine is a conditionally essential amino acid that is well absorbed, enhances wound healing, and modulates the function of neutrophils, macrophages, and lymphocytes. It also serves as a substrate for production of the antioxidant glutathione. During periods of catabolic stress (trauma, sepsis, burn), glutamine is released from muscle stores into the serum and intracellular levels of glutamine in muscle decrease. The tissues, particularly in splanchnic organs, rapidly take up the glutamine and glutamine serum concentrations subsequently fall. Previous work has demonstrated the efficacy of glutamine containing solutions in preventing peritoneal adhesion formation in a rat model. See U.S. Pat. No. 9,011,883.

In addition, glutamine is safe, well absorbed, and has no documented side effects. Glutamine is known to enhance wound healing. Glutamine and its dipeptides have been used for parenteral and enteral supplementation components in critically ill patients and in other clinical settings. The first uses of sources of glutamine have been employed either through enteral administration (e.g., in a food supplement) or parenteral administration through the intravenous route. At least one study described administration of glutamine to highly vascular areas, such as the peritoneum, where it is readily absorbed by the vasculature. At these locations, L-glutamine prevented the loss of vascularization due to peritoneal suturing that causes devascularization and adhesion formation. Thus, it was thought that glutamine may prevent fibrosis and adhesion formation by uptake of glutamine in the vasculature and preventing the loss of vascularization. As described herein, it was discovered that the application of a glutamine source to peripheral nerve or tissues surrounding the peripheral nerve significantly reduces fibrosis and associated adhesion.

Some embodiments described herein are methods of treating or reducing adhesion formation, fibrosis, or hypoxia-associated tissue damage a subject that is undergoing a surgical procedure. Exemplary and non-limiting surgical procedures contemplated herein include surgeries and procedures that affect the subject's head or neck, pelvic cavity, abdominal cavity, thoracic cavity, or one or more of the subject's limbs. Examples of surgeries that affect the pelvic cavity include, but are not limited to myomectomy, oophorectomy, hysterectomy, removal of endometriosis, tubal ligations, in short, any laparoscopic, laparotomic or open surgeries or procedures (including intravaginal procedures of the uterus, such as dilation and curettage, IVF, etc.) involving any of the reproductive organs, including but not limited to the uterus, ovaries, tubes, prostate, urethra, as well as the urinary bladder, the pelvic colon, and the rectum, or any surgeries involving the major arteries, veins, muscles, and nerves, membranes, ligaments or viscera of the pelvic cavity. Examples of surgeries that affect the abdominal cavity include but are not limited to gall bladder removal, liver resection, lap band surgery, anastomosis of the colon, appendectomy, in short, any surgeries involving the stomach, liver, pancreas, spleen, gallbladder, kidneys, and most of the small and large intestines any surgeries involving the major arteries, veins, muscles, and nerves, membranes, ligaments or viscera of the abdominal cavity. Examples of surgeries that affect the thoracic cavity include, but are not limited to, laparoscopic or laparotomic or open cardiovascular surgeries and procedures, lung surgeries, liver surgeries, gall bladder surgeries, any surgeries involving the major arteries, veins, muscles, and nerves, membranes, ligaments, bones or viscera of the thoracic cavity. Examples of surgeries that affect one or more of the subject's limbs include, but are not limited to laparoscopic or laparotomic or open surgeries involving the arms, legs, elbows, shoulders, spine, including, but not limited to any surgeries involving the major arteries, veins, muscles, and nerves, membranes, ligaments, bones or viscera of the, Examples of surgeries that affect the head and neck include but are not limited to laparoscopic or laparotomic or open surgeries and procedures involving the brain eye surgery or procedures, ear, nose and throat, teeth, gums, and jaw surgery or procedures, cosmetic reconstruction surgeries and procedures of the face, head and neck, cosmetic procedures to the teeth, gums and jaw any surgeries involving the major arteries, veins, muscles, and nerves, membranes, ligaments, bones or viscera of the head and neck.

In one embodiment, the source of glutamine is administered to a subject prior to, at the same time, or following a surgery involving one or more peripheral nerves. The prior administration results in the one or more peripheral nerves being protected from incurring fibrosis and eventual adhesion. Prior administration of the source of glutamine in some cases may further reduce or treat joint inflammation and recovery time following surgery in a subject. In another embodiment, the source of glutamine is administered to a subject after having undergone a surgery that involves one or more peripheral nerves. Similarly, one or more of the peripheral nerves are protected from undergoing additional fibrosis and adhesion. It is further contemplated that the administration of a source of glutamine can reduce adhesion.

In another embodiment, the source of glutamine is administered after the subject has sustained an injury to one or more peripheral nerves. The injury to the peripheral nerves may occur from an accident, a fall or sports can stretch, compress, crush or cut nerves; medical conditions, such as diabetes, Guillain-Barre syndrome and carpal tunnel syndrome; autoimmune diseases including lupus, rheumatoid arthritis and Sjogren's syndrome; and other causes include narrowing of the arteries, hormonal imbalances and tumors, cancer treatments including chemotherapy or radiotherapy.

In some embodiments, a subject has previously had a surgery or injury to one or more peripheral nerves and is administered a source of glutamine and has a reduction in the amount of peripheral nerve fibrosis and/or adhesion. In some embodiments, the amount of reduction of fibrosis and adhesion in the peripheral nerve of a subject is at least 10% in a subject following administration of the source of glutamine. In some embodiments the amount of reduction of adhesion within the subject having a previous surgery or injury is at least about 20%, 30%, 40%, 50%, 60%, 70%, 80%, 90%, 99%, or even 100%.

In some embodiments, one or more pathological conditions of one or more peripheral nerves or one or more tissues adjacent to or surrounding the peripheral nerve comprising fibrosis, inflammation, angiogenesis, immune cell infiltration, scarring, reduced joint flexibility, joint pain, or a combination of conditions thereof is reduced by an administration of a source of glutamine to one or more of the peripheral nerves or one or more tissues surrounding the one or more peripheral nerves of a subject.

In some embodiments the amount of reduction of one or more pathological condition of a peripheral nerve is decreased by at least about 20%, 30%, 40%, 50%, 60%, 70%, 80%, 90%, 99%, or even 100%. These pathological conditions of the peripheral nerve may be assessed by clinical tests known in the art.

In some embodiments, the source of glutamine is administered to one or more peripheral nerves in a manner to increase the residence time of glutamine at the peripheral nerve or one or more tissues surrounding the peripheral nerve. Without being bound by any theory, it is thought that increasing the time that glutamine is in contact with a peripheral nerve or tissue that is susceptible to undergoing adhesion or fibrosis events reduces the amount of adhesion or fibrosis to a greater extent. Glutamine intrinsically has a high turnover and is quickly absorbed by immune cells including lymphocytes and macrophages.

In some embodiments, the source of glutamine is administered to one or more peripheral nerve in a single dose, wherein an effective amount of glutamine is retained at or near the administration site for at least about 30 minutes to about 48 hours. In some embodiments, an effective amount of glutamine following a single administration is retained at or near the administration site for at least about 1 hour, about 2 hours, about 4 hours, about 6 hours, about 8 hours, about 10 hours, about 12 hours, about 16 hours, about 20 hours, about 24 hours, about 28 hours, about 32 hours, about 36 hours, about 40 hours, about 44 hours, or about 48 hours.

In some embodiments, the effective amount of a source of glutamine to reduce adhesion or one or more pathological conditions of the peripheral nerve described herein is administered in a single dosage. In some embodiments, the amount of a source of glutamine administered in a single dosage form ranges from about 0.0001 g/kg to about 5 g/kg of a subject's body weight. In some embodiments, the amount of a source of glutamine administered in a single dosage form is about 0.0001 g/kg, about 0.001 g/kg, about 0.01 g/kg, about 0.05 g/kg, about 1 g/kg, about 1.5 g/kg, about 2 g/kg, about 2.5 g/kg, about 3 g/kg, about 3.5 g/kg, about 4 g/kg, about 4.5 g/kg, about 5 g/kg. The exact amount of a source of glutamine administered to a subject will vary depending on the surgery or injury incurred the peripheral nerve being treated and the amount of pre-existing fibrous or adhesion and history of the subject. Because glutamine is an innocuous amino acid, large amounts may be administered without any expected side effects.

Some embodiments described herein are pharmaceutical dosage forms for reducing peripheral nerve fibrosis and adhesions in a subject. Some embodiments are methods of treating subject in need thereof with the pharmaceutical dosage forms described herein. Suitable pharmaceutical dosage forms include a source of glutamine and other pharmaceutically acceptable carriers and excipients for administration to the tissues or tissues surrounding the peripheral nerve.

In some embodiments, the glutamine source includes glutamine or any pharmaceutically acceptable salt thereof. In some embodiments, the source of glutamine includes D-glutatime or is D-glutamine, or L-glutamine or is L-glutamine. It is known that L-glutamine has a relatively low water solubility (i.e., about 40 g/L at room temperature) and low stability during storage.

Therefore, the source of glutamine may also include further carrier amino acids or glutamine can be incorporated as part of an oligopeptide, which may increase one or both of the solubility of the L-glutamine and stability. The oligopeptide may include any naturally occurring or non-naturally occurring amino acid. Suitable oligopeptides comprise L-glutamine and are capable of being metabolized to provide L-glutamine. Preferably, such peptides will exhibit increased aqueous solubility and increased stability of L-glutamine. Often, such peptides will also exhibit increased resistance to breakdown during sterilization and storage. The oligopeptide comprising L-glutamine may further include cleavable linker peptide moieties known in the art. These cleavable linkers may be utilized for attachment to various scaffolds and implants and for the administration methods described herein. In some embodiments, the source of glutamine is L-glutamine incorporated as part of a dipeptide. Two such peptides that may be used are dipeptides comprising L-glutamine and either L-alanine or glycine. The dipeptide alanyl-glutamine (glutamine residue at the C-terminal position) has high solubility in water (568 g/L). Glycyl-glutamine (glutamine at the C-terminal position) also shows enhanced solubility in water as compared to glutamine (154 g/L). Each of the latter di peptides is sufficiently stable during heat sterilization and prolonged storage that they have been previously employed in total parenteral nutrition formulations for intravenous injection. Thus, in one embodiment, the source of glutamine comprises L-alanyl-L-glutamine. In other embodiments, the source of glutamine comprises is D-glutamine incorporated as part of a dipeptide. Two such peptides that may be used are dipeptides comprising D-glutamine and either D-alanine or glycine. Thus, in one embodiment, the source of glutamine comprises D-alanyl-D-glutamine. In some embodiments, the source of glutamine is provided as part of an oligopeptide comprising glutamine, wherein an aqueous solubility and an aqueous stability of the source of glutamine is higher than glutamine alone. In other embodiments, the glutamine source is a glutamine conjugate wherein at least one glutamine residue is bound to a compound via an amino ester bond. Examples of such glutamine sources include, but are not limited to, dichloroacetylglutamine, acetylglutamine, butryrlglutamine, pyruvylglutamine, and glutamine conjugated to any other suitable organic acid via an amino ester bond.

L-alanyl-L-glutamine is a dipeptide known for its ability to promote wound healing, immune modulation and regulation of ECM matrix and peritoneal fibrosis. We believe hypoxia in fibrotic tissues could be chronic and acute depending on the extent of injury and tissue damage. Chronic hypoxia is hypothesized to be present at the exact site of injury, which we have represented as consistent or continuous hypoxia. Whereas acute hypoxia could be present at the peripheral ends from the exact site of injury represented by irregular or episodic hypoxia. While not being bound by any particular theory, the inventors surprisingly found that administering a glutamine source, such as L-alanyl-L-glutamine, to hypoxic primary rat fibroblasts dramatically downregulates HIF1-α. In addition, as HIF1-α modulates other pro-fibrotic factors including SMAD signaling, the inventors found that the glutamine source was able to downregulate these factors as well. Accordingly, the inventors have found that the administering a glutamine source Our observations led us to conclude that L-Alanyl-L-Glutamine regulates both of modes of hypoxic injury (chronic and episodic).

Pharmaceutically acceptable preparations of L-glutamine and L-glutamine containing peptides (including L-alanyl-L-glutamine) are commercially available. In addition, L-glutamine containing peptides for use in the methods described herein may also be synthesized according to known methodology and purified and sterilized for pharmaceutical use. Other pharmaceutically acceptable may be prepared according to embodiments of the present invention, including but not limited to, preparations of D-glutamine and D-glutamine containing peptides (including D-alanyl-D-glutamine).

In some embodiments, the source of glutamine is administered with one or more additional active pharmaceutical ingredients. The additional active pharmaceutical ingredient may be administered in the same way as the source of glutamine or by a different suitable parenteral method or enteral method. In some embodiments, the additional one or more active pharmaceutical ingredients is administered with the source of glutamine by the same route of administration prior to, at the same time, or following the administration of the source of glutamine. In other embodiments, the additional one or more active pharmaceutical ingredients are administered with the source of glutamine by a different route of administration prior to, at the same time, or following the administration of the source of glutamine. Suitable pharmaceutically active ingredients include any drug or agent that is used for treating joint pain, inflammation, or adhesion. Exemplary and non-limiting drugs include non-steroidal anti-inflammatory drugs (NSAID) aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, meloxicam, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, tolmetin, or valdecoxib; selective COX-2 inhibitors, such as celecoxib, rofecoxib, prostaglandin; anti-arthritic supplements, such as hyaluronic acid, chondroitin sulfate, and glucosamine; a corticosteroid, such as betamethasone, methylprednisolone, and triamcinolone. The source of glutamine may further be administered with an anti-autoimmune disease drugs such as disease modifying anti-rheumatic drugs (DMARDs) including but not limited to anti-folates, methotrexate, leflunomide, mycophenolate mofetil, sulfasalazine, apremilast, tofacitinib; biologics, such as tocilizumab, certolizumab, etanercept, adalimumab, anakinra, abatacept, infliximab, and rituximab.

In some embodiments, the source of glutamine is administered by intra-articular injection, topical administration, transdermal administration, by iontophoresis, or surgically.

In some embodiments, the source of glutamine is formulated for delivery to a peripheral nerve or tissue surrounding a peripheral nerve by injection comprising an intraarticular injection method known in the art. In one embodiment, the glutamine source (e.g., L-alanyl-L-glutamine) can be formulated as part of an aqueous solution with a pharmaceutically acceptable carrier such as sterile distilled water, sterile isotonic solutions, sterile physiological saline solutions or dry buffer and/or salt mixes or concentrations which when diluted form such a solution.

In some embodiments, the formulations may be a liquid, paste, microparticle (including, but not limited to, liposomes and other vesicles) or gel comprising a glutamine source dissolved in an aqueous phase. Compositions described herein may be such formulations or may be compositions intended to produce such a formulation when hydrated. In its simplest form, a formulation for use in this invention will consist of a glutamine source dissolved in a sterile aqueous liquid vehicle, suitable for instilling within a peripheral nerve during surgery or for interarticular injection performed thereafter. The formulation may be instilled through a port created for laparoscopic surgery or during laminectomy.

In some embodiments, the source of glutamine is formulated as part of a gel or hydrogel. Hydrogel formulations allow for an extended delivery of suitable drugs including for example, L-glutamine or di- or tri-peptides containing at least one glutamine residue. Hydrogels typically form a depot, which further allows for a concentrated application of a drug. The hydrogel comprising a source of glutamine may be generated prior to administration and impregnated with the source of glutamine for further implantation. Alternatively, hydrogels that form in situ after change environment, including pH and temperature may be utilized for intra-articular injection. Various physical and chemical cross-linking polymers for in situ hydrogel formation are known in the art. Exemplary and non-limiting hydrogels may include co-polymers comprising blocks of poly ethylene oxide (PEO) propylene oxide (PPO), poly(lactide-co-glycolic acid) (PLGA), poly (Nisopropylacry lamide), poly(propy lene fumarate), poly(caprolactone) and the like. Suitable hydrogels may also be generated from naturally occurring proteins and peptides. The particular hydrogel formulation can be determined by the application site or the specific joint being treated.

In some embodiments, the source of glutamine is formulated as part of an implantable film containing the source of glutamine. The glutamine source may be applied to or impregnated in the surgical implantable film or other surgical implant. For example, the source of glutamine may be formulated as part of a gel and adhered to the exterior of an implant. Implants composed of a material such as woven resorbable cellulose commercially available as INTERCEED® from Ethicon may be impregnated with a liquid or gel formulation of this invention. Other films contemplated include polyesteramide based films (PEA-III) (see, for example, PCT International Application Publication No. WO/2014053542Al). In some other embodiments, the source of glutamine is formulated for topical, transdermal, or for iontophoretic administration.

In some embodiments, the drug load of the source of glutamine within the dosage form is about 2% to about 90%, including each integer within the specified range. In some embodiments, the drug load is about 10% to about 80%. In some embodiments, the drug load is about 20% to about 60%. In some embodiments, the drug load is about 20% to about 50%. In some embodiments, the drug load is about 20% to about 40%. In some embodiments, the drug load is about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, the drug is impregnated into an implantable film. In some embodiments, the drug load in the implantable film is about 2% to about 90%, including each integer within the specified range. In some embodiments, the drug load in the implantable film is about 10% to about 80%. In some embodiments, the drug load in the implantable film is about 20% to about 60%. In some embodiments, the drug load in the implantable film is about 20% to about 50%. In some embodiments, the drug load in the implantable film is about 20% to about 40%. In some embodiments, the drug load in the implantable film is about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%. In one embodiment the drug load in the implantable film is about 30%. In another embodiment the drug load in the implantable film is about 50%.

In some embodiments, the dosage form comprising a source of glutamine is stable for months or years. In some embodiments, the pharmaceutical dosage forms of a source of glutamine described herein are stable at 25° C. and 60% relative humidity (RH) for about 1 month, about 2 months, about 3 months, about 4 months, about 5 months, about 6 months, about 9 months, about 10 months, about 11 months, about 12 months, or even longer.

Some embodiments described herein are kits for preventing or reducing peripheral nerve fibrosis or adhesion in a subject. The kits described herein include a source of glutamine (e.g., L-alanyl-L-glutamine) in a suitable dosage form described herein. The kits described herein further include instructions and suitable administration schedules depending on the peripheral nerve being treated. In one embodiment, the kit comprises a dosage form that is provided as an injectable. In another embodiment, the dosage form comprises a dosage form that is a film impregnated with the source of glutamine. In another embodiment, the kit comprises a dosage form that is a topical cream. In another embodiment, the kit comprises a dosage form that is suitable for iontophoretic delivery. In some embodiments, the kit comprises multiple dosage forms for administration to a subject in need thereof.

### Examples

A study investigated the ability of L-Alanyl-L-Glutamine to regulate HIF1a mediated fibrotic response of hypoxic primary fibroblasts isolated and purified from rat sciatic nerves.

### Isolation and Culture of Rat Fibroblasts

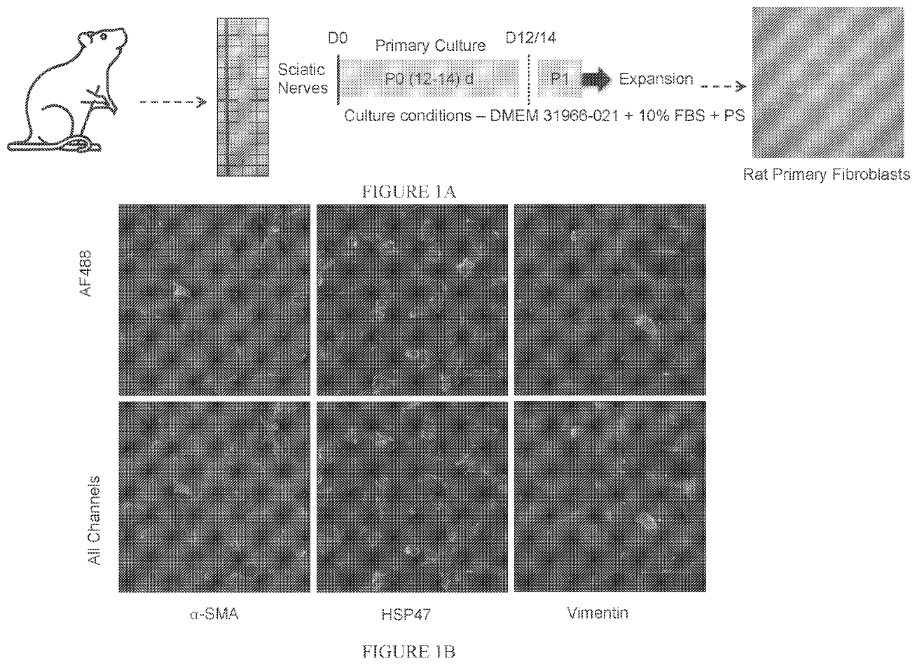

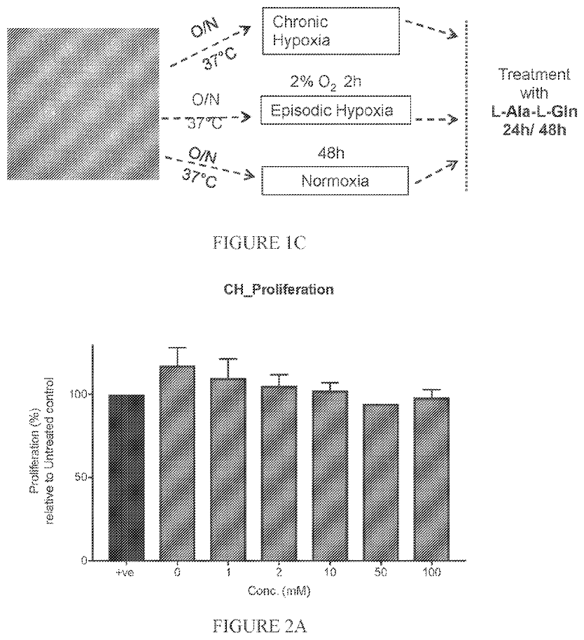

The local veterinary commission and the cantonal veterinary office approved our study and animal protocol including euthanizing, harvesting of sciatic nerves and its further use for isolation of primary fibroblasts. Adult Sprague-Dawley rats were euthanized by using a CO2 gas chamber and sciatic nerves were dissected out immediately under aseptic conditions and placed in ice-cold Dulbecco's Phosphate Buffered Saline (DPBS) (Sigma-Aldrich). Each adult sciatic nerve was cleaned off excess tissue including muscle, fat and blood vessels while working under a Stereo dissecting microscope (Carl Zeiss Miscroscopy GmbH, Germany). Subsequently the epineurium was stripped off with the help of fine forceps. Followed by this, we dissected the nerve segments into small sections (2-5 mm) and placed 5-6 segments in 6-well plates supplemented in culture medium consisting of Dulbecco's Modified Eagle Medium (DMEM) (Gibco) with 10% fetal bovine serum (FBS) (BI) and 1% pen/strep (Gibco) for 12-14 days. Medium was typically replaced every 2-3 days. Primary fibroblasts sprouted from the nerve tissues (FIG. 1A) and once cells approached confluency, tissue segments were removed from the 6-well plates with the help of forceps. Cells were washed twice with ice-cold PBS to remove tissue debris, trypsinized with 0.25% Trypsin (Gibco), followed by centrifugation at 1800 rpm for 5 minutes. Cells were counted using Trypan blue and plated in T25/T75 culture flasks (Corning) until for further experimentation. Prior to experimentation, all cells isolated were stained for α-SMA, HSP47 and Vimentin, to confirm the phenotype of these cells (FIG. 1B). All experiments with primary rat fibroblasts were performed from passages 1-5.

### Hypoxia and L-Alanyl-L-Glutamine Treatment

In order to mimic the hypoxic micro-environment, primary rat fibroblasts were exposed to hypoxia. Cells were exposed to 2 separate conditions—continuous and episodic hypoxia. For continuous hypoxia, cells were incubated in a hypoxic chamber (2% 02) for 48 h and treated with or without L-alanyl-L-glutamine and no restoration to normoxia as shown in FIG. 1C. Cells were collected and processed for experiments subsequently.

For episodic hypoxia, cells were incubated in a hypoxic chamber (2% 02) for 2 h followed by restoration to normoxia for 48 h at which time cells were treated with or without L-alanyl-L-glutamine, as shown by FIG. 1C. Cells were collected after 48 h and processed for experiments subsequently.

### Proliferation Assays

Proliferation of primary fibroblasts were determined by alamar blue assay (Invitrogen). Briefly, cells were plated in a 96-well plate treated with or without L-alanyl-L-glutamine under aforementioned hypoxic conditions for 48 h. The interaction of alamar blue with the cells was analyzed for 4 h by measuring fluorescence readings at 540 nm and 585 nm respectively.

Proliferation was also detected using flow cytometry based EdU assay using EdU-Click 488 (baseclick).

### Cell Death Assay

Effect of L-alanyl-L-glutamine on cell death of hypoxic fibroblasts were determined by Annexin V/7-AAD (BD Pharmingen) using flow cytometry. For the cell death assay, adherent fibroblasts were cultured under hypoxic conditions and treated with and without L-alanyl-L-glutamine at various concentrations. Cells were collected after 48 h and washed and stained with Annexin V and 7-AAD and analyzed using flow cytometry.

### Western Blotting

Protein samples were prepared by resuspending cells growing in 6-well plates in RIPA lysis and extraction buffer (Thermo) containing protease and phosphatase inhibitors (Roche). Protein concentrations were quantified using protein assay dye reagent (Bio-Rad). Samples were mixed with 4× LDS Sample buffer (Thermo) and denatured by boiling at 95° C. for 6 minutes. Following protein concentration estimations, equal protein samples containing 25 μg total protein were loaded and resolved in 4-15% Mini protean TGX gels (Bio-Rad). Fractionated proteins were transferred to 0.2 μm nitrocellulose membranes using Trans-Blot® Turbo™ Transfer System (Bio-Rad). Membranes were blocked with 5% Non-Fat dry milk and incubated overnight with primary antibodies.

The following primary antibodies along with their respective concentrations were used during overnight incubation. Anti-collagen I (1:500; ab34710) and anti-collagen IV (1:500; ab6586) were purchased from Abcam. Anti-heat shock protein 47 (HSP-47) (1:400; sc-13150) and anti-fibronectin (1:500; sc-8422) were ordered from Santa Cruz Biotechnology. Other antibodies including anti-SMAD 2/3 (1:500; 85865S), anti-GAPDH (1:5000; 97166S) and anti-HIF-1α (1:750; 14179S) were from Cell Signaling Technology.

Horseradish peroxidase-conjugated secondary antibodies at a concentration of 1:5000 were used for immunoreactivity and detection of protein bands were does using Pierce™ ECL Plus Western Blotting Substrate (Thermo) and on the ChemiDoc Imaging Systems (Bio-Rad).

### Immunofluorescence

For immunofluorescence microscopy, cells were grown on 4-well chamber slides (IBIDI) under appropriate hypoxic conditions with or without L-alanyl-L-glutamine treatment. Cells were washed with ice-cold PBS followed by fixation with 4% paraformaldehyde. Fixed cells were permeabilized with 0.5% Triton X-100 and incubated with blocking reagent (1% BSA in PBS) for 30 minutes, prior to overnight incubation with appropriate primary antibodies. After wash steps, cultures were labeled and incubated with secondary antibodies and 4′-6-didiamino-2-phenylindole, dihydrochloride (DAPI, Sigma) were used to stain the nuclei.

Fluorescence microscopy image analysis was performed using NIKON Ti2 inverted microscope equipped with Photometrics Prime 95B camera for fluorescence imaging. All images were digitally processed, all quantifications and further arrangement for presentation was done using NIS-Elements Advanced Research analysis software. For quantification analysis, pictures from random fields in independent wells were taken at 10× and 40× magnification objectives and the total protein expression for each marker was determined in reference to the total number of cells based on DAPI quantification.

For immunofluorescence, the primary antibodies along with their respective concentrations are listed below. Anti-collagen I (1:500; ab34710), anti-collagen IV (1:500; ab6586) and anti-α-SMA (1:500; ab124964) were purchased from Abcam. Anti-HSP-47 (1:250; sc-13150), anti-fibronectin (1:250; sc-8422), and anti-HIF-1α (1:500; sc-13515) were ordered from Santa Cruz Biotechnology. Anti-SMAD 2/3 (1:500; 85865S) was purchased from Cell Signaling Technology. In addition to this, the following secondary antibodies: Goat anti-mouse Alexa Fluor (AF) 488 (A11029), goat anti-mouse AF546 (A11030), goat anti-rabbit AF488 (A11034) and goat anti-rabbit AF546 (A11010) from Life Technologies. DAPI (D9564) from Sigma was used at a concentration of 1:1000.

### Sample Preparation for Phosphoproteomics Analysis

Following fibroblast isolation from sciatic nerves of rats, (8E10 primary fibroblasts) were lysed in 80 ul of 8M Urea, 0.1M ammonium bicarbonate, phosphatase inhibitors (Sigma P5726&P0044) by sonication (Bioruptor, 10 cycles, 30 seconds on/off, Diagenode, Belgium) and proteins were digested as described previously (PMID:27345528). Shortly, proteins were reduced with 5 mM TCEP for 60 min at 37° C. and alkylated with 10 mM chloroacetamide for 30 min at 37° C. After diluting samples with 100 mM ammonium bicarbonate buffer to a final urea concentration of 1.6M, proteins were digested by incubation with sequencing-grade modified trypsin (1/50, w/w; Promega, Madison, Wisconsin) for 12 h at 37° C. After acidification using 5% TFA, peptides were desalted using C18 reverse-phase spin columns (Macrospin, Harvard Apparatus) according to the manufacturer's instructions, dried under vacuum and stored at −20° C. until further use.

Peptide samples were enriched for phosphorylated peptides using Fe(III)-IMAC cartridges on an AssayMAP Bravo platform as recently described (PMID: 28107008). Phospho-enriched peptides were resuspended in 0.1% aqueous formic acid and subjected to LC-MS/MS analysis using a Q-Exactive plus mass spectrometer fitted with an EASY-nLC 1000 (both Thermo Fisher Scientific) and a custom-made column heater set to 60° C. Peptides were resolved using a RP-HPLC column (75 μm×30 cm) packed in-house with C18 resin (ReproSil-Pur C18-AQ, 1.9 μm resin; Dr. Maisch GmbH) at a flow rate of 0.2 μL/min. The following gradient was used for peptide separation: from 5% B to 8% B over 5 min to 20% B over 45 min to 25% B over 15 min to 30% B over 10 min to 35% B over 7 min to 42% B over 5 min to 50% B over 3 min to 95% B over 2 min followed by 18 min at 95% B. Buffer A was 0.1% formic acid in water and buffer B was 80% acetonitrile, 0.1% formic acid in water.

Mass spectrometry analysis was performed on Q-Exactive plus mass spectrometer equipped with a nanoelectrospray ion source (both Thermo Fisher Scientific). Each MS1 scan was followed by high-collision-dissociation (HCD) of the 10 most abundant precursor ions with dynamic exclusion for 20 seconds. Total cycle time was approximately 1 s. For MS1,3e6 ions were accumulated in the Orbitrap cell over a maximum time of 100 ms and scanned at a resolution of 70,000 FWHM (at 200 m/z). MS2 scans were acquired at a target setting of 1e5 ions, accumulation time of 50 ms and a resolution of 17,500 FWHM (at 200 m/z). Singly charged ions and ions with unassigned charge state were excluded from triggering MS2 events. The normalized collision energy was set to 27%, the mass isolation window was set to 1.4 m/z and one microscan was acquired for each spectrum.

The acquired raw-files were imported into the Progenesis QI software (v2.0, Nonlinear Dynamics Limited), which was used to extract peptide precursor ion intensities across all samples applying the default parameters. The generated mgf-files were searched using MASCOT against a decoy database containing normal and reverse sequences of the predicted (Proteomes) UniProt entries of Rattus norvegicus (www.uniprot.org, release date 2020/03/10) and commonly observed contaminants (in total 60,690 sequences) generated using the SequenceReverser tool from the MaxQuant software (Version 1.0.13.13). The search criteria were set as follows: full tryptic specificity was required (cleavage after lysine or arginine residues, unless followed by proline); 3 missed cleavages were allowed; carbamidomethylation (C) was set as fixed modification; oxidation (M) and phosphorylation (STY) were applied as variable modifications; mass tolerance of 10 ppm (precursor) and 0.02 Da (fragments). The database search results were filtered using the ion score to set the false discovery rate (FDR) to 1% on the peptide and protein level, respectively, based on the number of reverse protein sequence hits in the datasets. Quantitative analysis results from label-free quantification were processed using the SafeQuant R package v.2.3.2. (PMID:27345528, https://github.com/eahrne/SafeQuant/) to obtain peptide relative abundances. This analysis included global data normalization by equalizing the total peak/reporter areas across all LC-MS runs, data imputation using the knn algorithm, summation of peak areas per and LC-MS/MS run, followed by calculation of peptide abundance ratios. Only isoform specific peptide ion signals were considered for quantification. The summarized peptide expression values were used for statistical testing of between condition differentially abundant peptides. Here, empirical Bayes moderated t-Tests were applied, as implemented in the R/Bioconductor limma package (http://bioconductor.org/packages/release/bioc/html/limma.html) were used. The resulting per protein and condition comparison p-values were adjusted for multiple testing using the Benjamini-Hochberg method.

### Statistical Analysis

Statistical analysis was performed with Prism 7.0 software. Experimental data and graphical plots are expressed as the mean±standard deviation (SD) of a particular experiment out of at least 3 independent experiments including 3 biological replicates, unless otherwise mentioned. Statistical significance of various conditions was characterized by the Student t-Test or one-way ANOVA. For multiple comparisons, we used Sidak's multiple comparisons test and statistical significance was considered for P values<0.05. Statistically significant values are shown with their degree significance—* (p<0.05), ** (p<0.01), *** (p<0.001) and **** (p<0.0001).

### Results

L-Alanyl-L-Glutamine does not Affect Cell Proliferation and Viability of Hypoxic Primary Fibroblasts

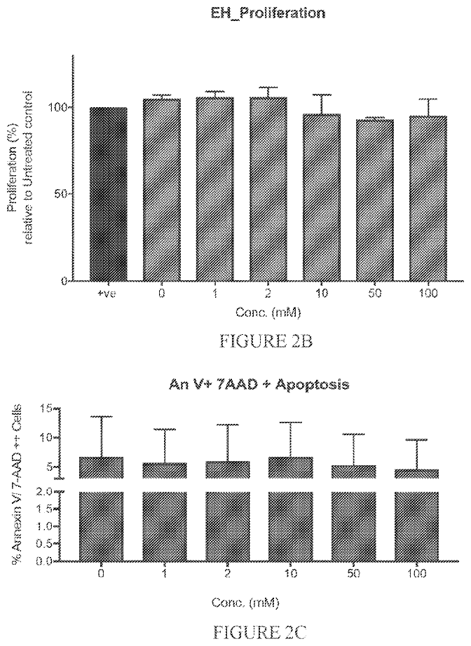

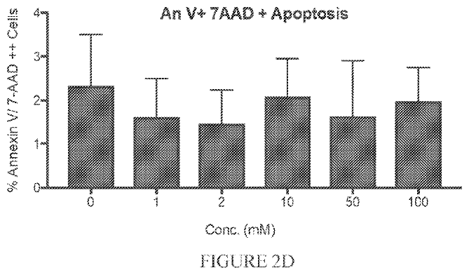

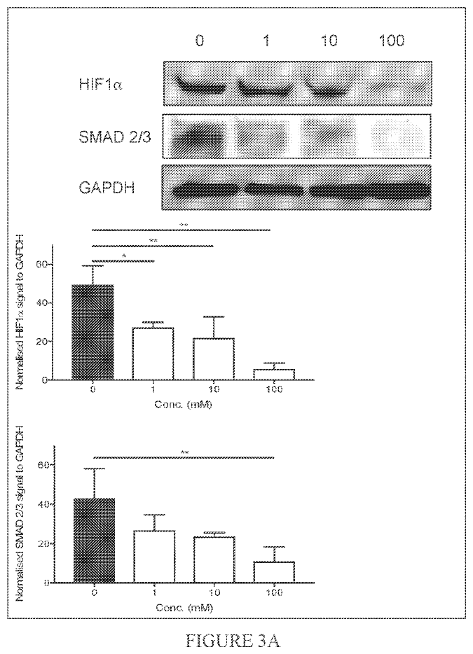

Hypoxia being a primary microenvironment in fibrotic tissues, we performed all our in vitro analysis using primary rat perineurium derived fibroblasts exposed to hypoxic conditions. To investigate whether L-alanyl-L-glutamine treatment affects the viability of primary rat fibroblasts, we examined viability over 48 h using the Alamar Blue assay. Our in vitro analysis showed that, L-alanyl-L-glutamine does not affect the viability and cell proliferation of hypoxic primary rat fibroblasts. None of the concentrations including 1 mM, 10 mM and 100 mM induced any significant changes on cell viability and proliferation. Compared with the untreated controls, primary rat fibroblasts exposed under continuous (48 h) and episodic (2 h) hypoxia exhibited no change in cell proliferation when treated with L-alanyl-L-glutamine under various concentrations, as shown in FIGS. 2A-2D.

### L-Alanyl-L-Glutamine Down Regulates Expression of Fibrotic Proteins of Primary Fibroblasts Exposed Under Continuous Hypoxia

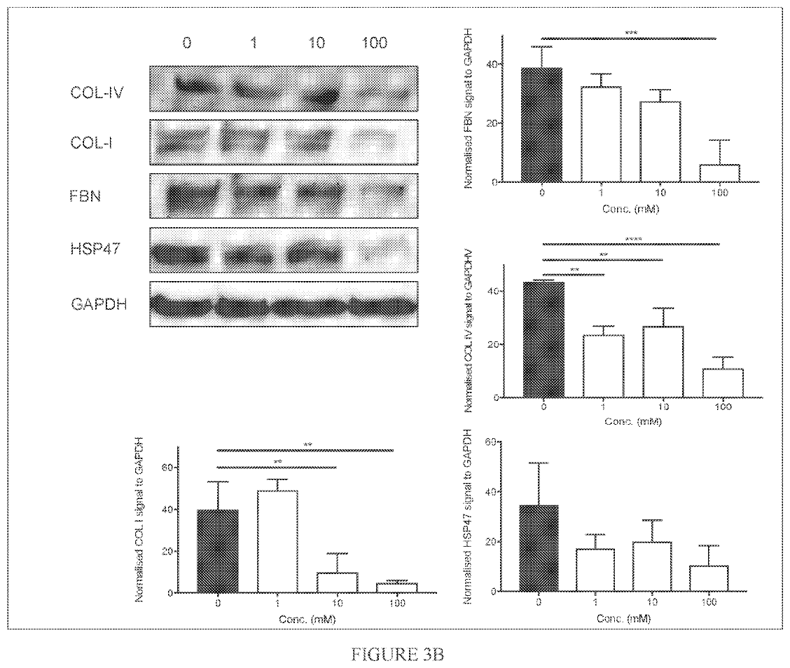

Hypoxia increases expression of pro-fibrotic and adhesion biomarkers in fibrotic diseases. To determine whether L-alanyl-L-glutamine induces any change on hypoxic microenvironment, we tested the expression of HIF1-α, under the treatment of various concentration of L-alanyl-L-glutamine. As shown in FIG. 3A, western blotting of whole cell lysates of hypoxic primary fibroblasts showed that L-alanyl-L-glutamine significantly down regulates markers involved with induction of hypoxic microenvironment, HIF1-α and SMAD 2/3. HIF1-α was significantly down regulated upon treatment with L-alanyl-L-glutamine at concentration of 1 mM, 10 mM and 100 mM. SMAD 2/3 was also significantly downregulated in hypoxic primary fibroblasts by 100 mM L-alanyl-L-glutamine only, as shown in FIG. 3A.

Additionally, we further investigated the effect of L-alanyl-L-glutamine on other pro-fibrotic factors. We observed that continuous hypoxia for 48 h induces the expression of pro-fibrotic markers including Collagen-IV, Collagen-I, and Fibronectin as compared to normoxic untreated controls. As shown in FIG. 3B, treatment with L-alanyl-L-glutamine at 1 mM, 10 mM and 100 mM significantly reduced the expression of these fibrotic proteins in a dose-dependent manner. As HYP47 is another key pro-fibrotic factor responsible for collagen biosynthesis, we tested our hypothesis on HSP47 and found that L-alanyl-L-glutamine reduces expression of HSP47 but we did not observe a significant downregulation, as shown in FIG. 3B.

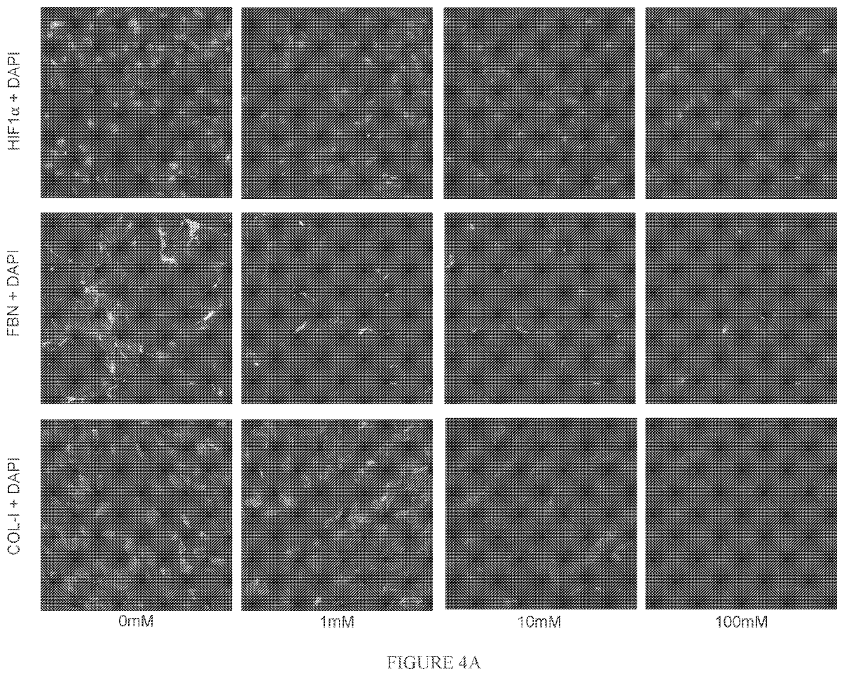

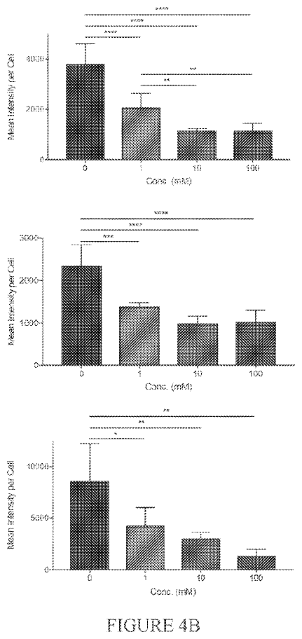

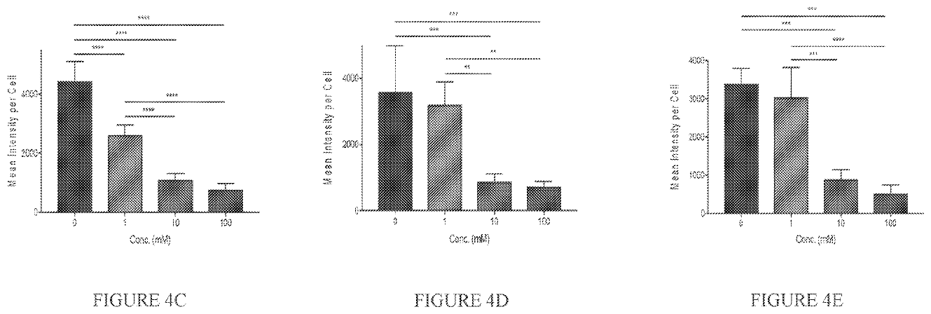

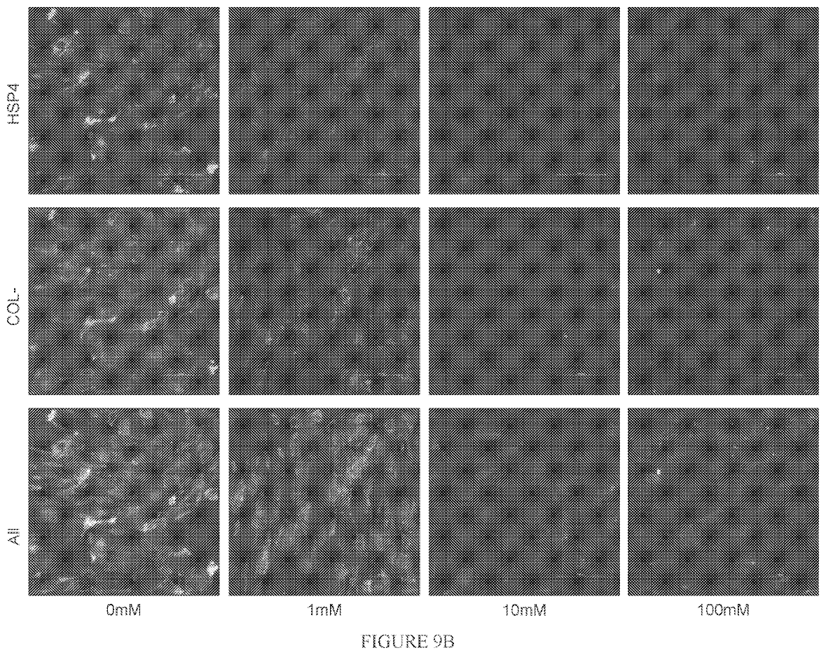

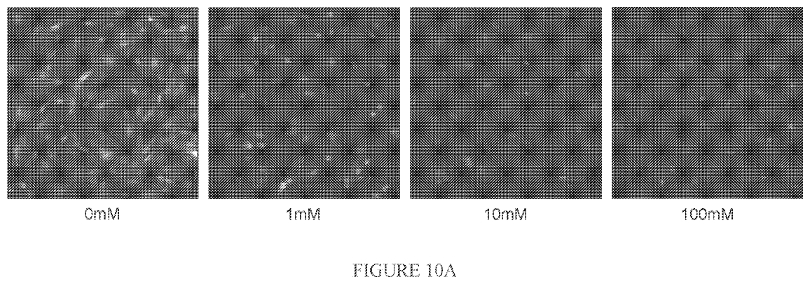

Immunofluorescence analysis of hypoxic primary fibroblast treated with or without L-alanyl-L-glutamine under various concentrations was also performed. In vitro immunofluorescence analysis revealed that treatment with L-alanyl-L-glutamine resulted in a significant reduction of factors responsible for cellular response to hypoxia including HIF1-a and SMAD 2/3, as shown in FIGS. 4A, 4B, 4C, and 9A. HIF1-a and SMAD 2/3 were stained in the nuclei and both were downregulated upon treatment with L-alanyl-L-glutamine.

Cytoplasmic staining of pro-fibrotic factors including Fibronectin, Collagen-IV, Collagen-I and HSP47 was also performed. We found these targets were accumulating in hypoxic primary fibroblasts, as shown in FIGS. 4A, 4B, 4D, 4E, and 9B, and correspondingly were significantly downregulated in L-alanyl-L-glutamine treated fibroblasts. Our statistical analysis revealed a dose-dependent downregulation of HIF1-α, SMAD 2/3, Collagen-IV, and HSP47 in L-alanyl-L-glutamine treated fibroblasts (FIG. 4A, 4C, 4D and 4E), but was significantly reduced in a non-dose dependent manner for Fibronectin and Collagen-I (FIGS. 4A and 4B). Further experiments to understand this regulation should be conducted to understand this behavior of L-alanyl-L-glutamine in reducing factors involved with collagen biosynthesis.

### L-Alanyl-L-Glutamine Down Regulates Expression of Fibrotic Proteins of Primary Fibroblasts Exposed Under Episodic Hypoxia

Overexpression of HIF1-α in fibrotic tissues can generate chronic or acute hypoxic microenvironments. The effects of L-alanyl-L-glutamine on pro-fibrotic factors in episodic hypoxic primary rat fibroblasts were tested. We investigated the effect of L-alanyl-L-glutamine in hypoxic primary fibroblasts exposed to episodic hypoxia for 2 h followed by treatment with or without L-alanyl-L-glutamine for 48 h.

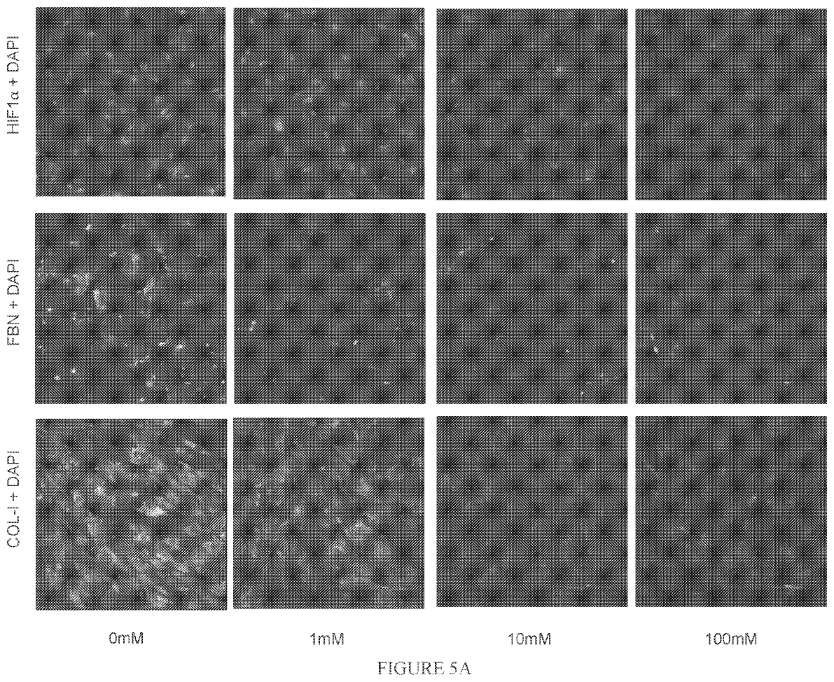

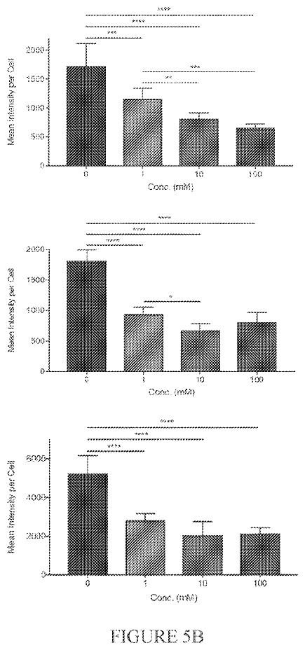

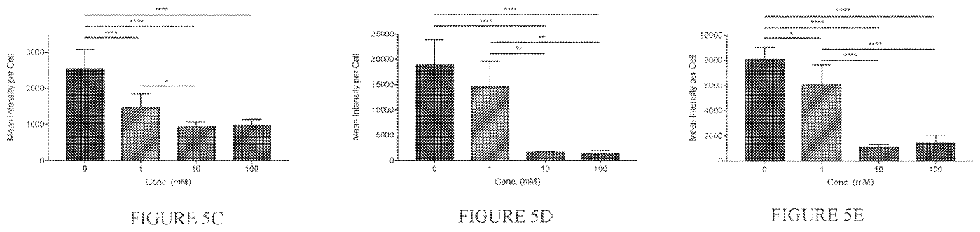

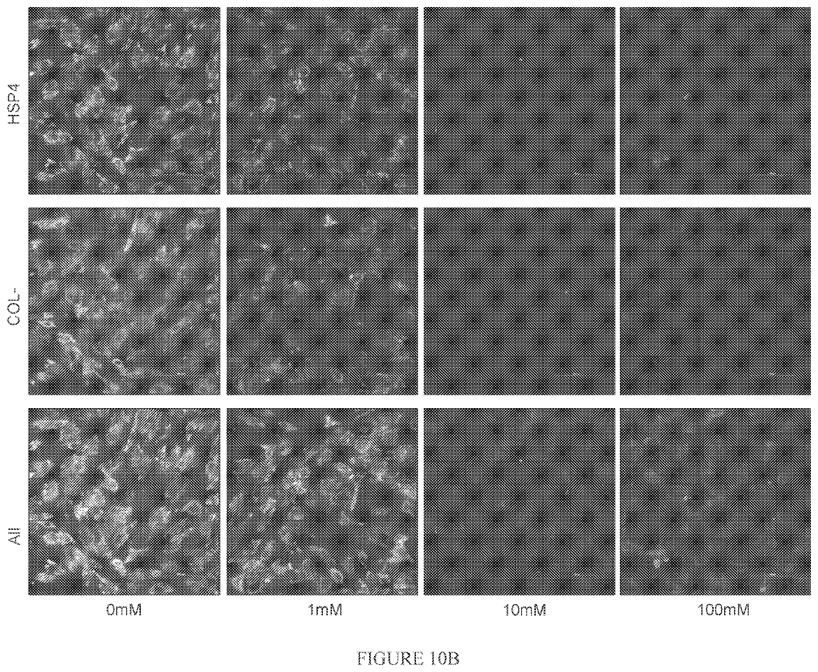

Intracellular staining of specific targets responsible for fibrogenic potential as well as proteins regulated in HIF1—a signaling was performed. The immunofluorescence analysis showed that L-alanyl-L-glutamine effectively reduces expression of pro-fibrotic proteins including Fibronectin, Collagen-I, Collagen-IV and HSP47, as shown in FIGS. 5A, 5B, 5D, 5E, and 10B. As shown in FIGS. 5A, 5B, 5C, and 10A, significant downregulation of downstream targets of HIF1—a signaling pathway namely HIF1—a and SMAD 2/3 in L-alanyl-L-glutamine were observed as well. Following immunocytochemistry, mean intensity per cell were calculated for these proteins in L-alanyl-L-glutamine treated samples and compared to untreated hypoxic samples. L-alanyl-L-glutamine significantly reduced expression of adhesion and fibrotic proteins in a dose-dependent fashion. As demonstrated in FIGS. 5B-5E All above factors were significantly upregulated in hypoxic samples (FIGS. 5B-E). We observed a dose-dependent downregulation of intra-expression of all the proteins except Collagen-I. Collagen I was found to be significantly reduced at 1 mM L-alanyl-L-glutamine, but remained constant thereafter (FIG. 5A, 5B; Supplementary 3B).

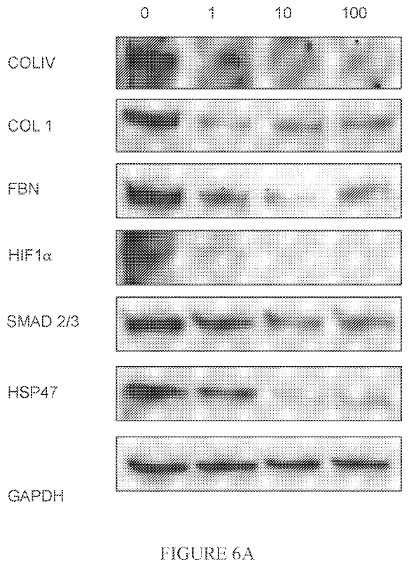

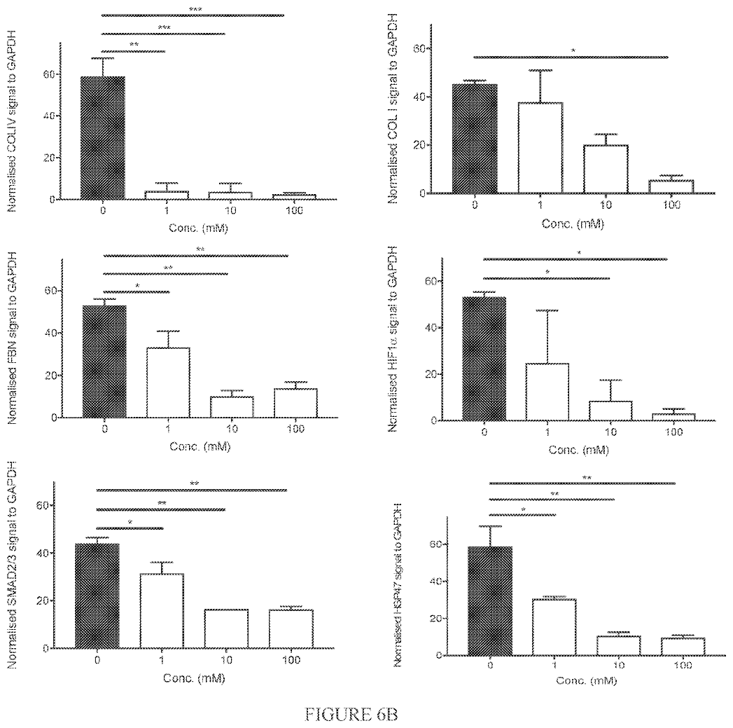

Western blotting on whole cell lysates of episodic hypoxic primary fibroblasts was also performed. In contrast to immunocytochemistry, the Western analysis did not reveal any dose-dependent regulation of our desired targets. HIF1-α signaling factors and pro-fibrosis mechanistic targets were significantly upregulated in hypoxic samples in comparison to L-alanyl-L-glutamine treated samples. As shown in FIG. 6B, there was not any significant downregulation across the various concentrations of L-alanyl-L-glutamine. Interestingly, we found significant downregulation of COL-IV in 1 mM L-alanyl-L-glutamine treated hypoxic fibroblasts, shown in FIG. 6B, which is slightly different from the immunofluorescence analysis, as shown in FIG. 5E).

Identification of crucial proteins and pathway analysis in hypoxic fibroblasts treated with L-alanyl-L-glutamine

This study is the first attempt to compile the phospho-proteome profile of L-alanyl-L-glutamine treated under chronic hypoxic conditions treated with the different concentrations of L-alanyl-L-glutamine. A total of 5110 proteins were profiled in all 4 conditions (0 mM, 1 mM, 10 mM and 100 mM). While 1322 proteins were found to be significantly regulated across the treatment group (1 mM, 10 mM and 100 mM) when compared with 0 mM hypoxic samples. Out of these 1322 proteins, 607 proteins were significantly downregulated and 715 were significantly upregulated across the L-alanyl-L-glutamine samples. Biological triplicates of each condition were used and a pooled analysis of all biological triplicates was performed between the treated group and the untreated 0 mM condition. Positive values of log_2 ratios indicate upregulation of associated protein in L-alanyl-L-glutamine treated group while negative values indicate downregulation. Further analysis for identification of critical factors and associated pathways were performed using DAVID statistical software and KEGG pathway analysis as described earlier. Analysis was focused on downregulated targets to identify proteins and associated pathways regulated by L-alanyl-L-glutamine to reduce fibrogenic phenotype. Pathway analysis showed that proteins significantly downregulated in the treated groups were associated with mTOR, PI3k, cell adhesion and Focal adhesion.

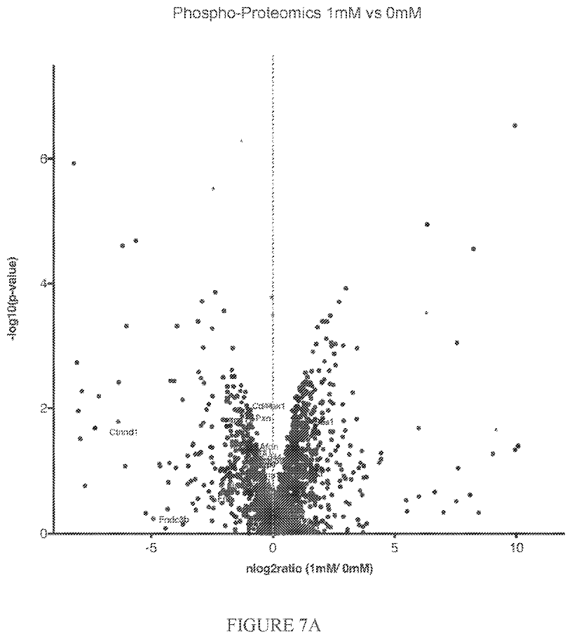

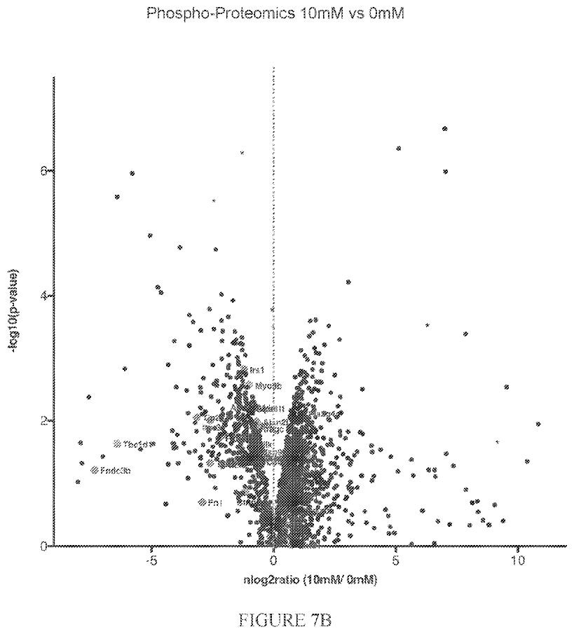

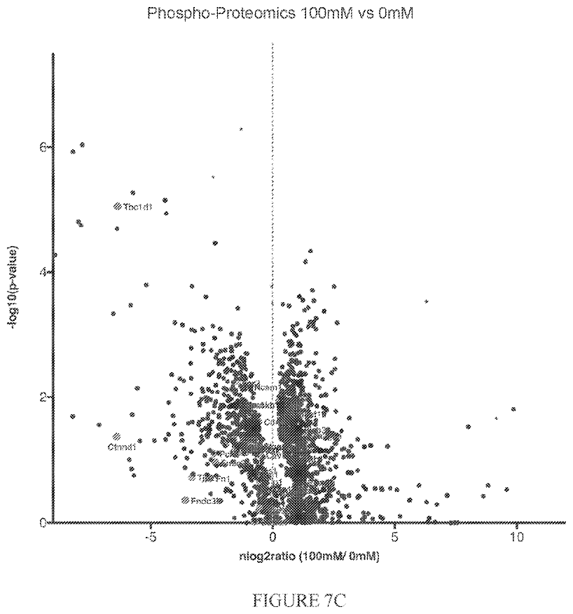

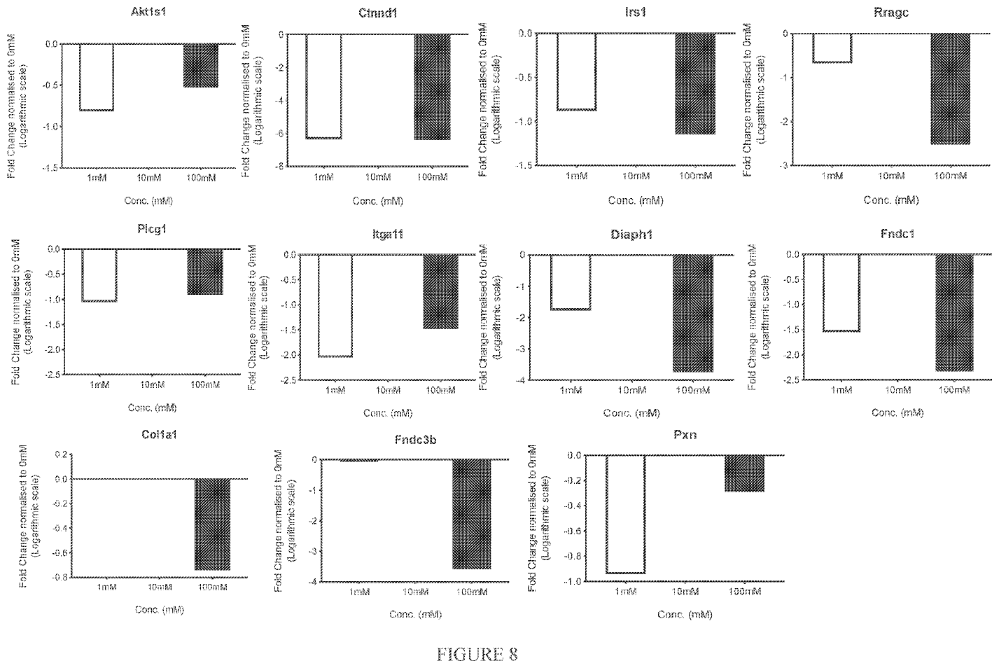

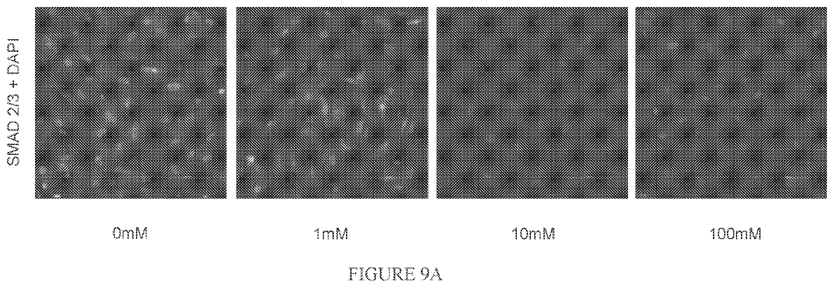

As shown in FIGS. 7A-7C, L-alanyl-L-glutamine effectively downregulates crucial factors involved with cell adhesion and fibrosis progression but we were also able to identify a dose-dependent effect of L-alanyl-L-glutamine in the expression of these factors. Several targets associated with the HIF1-α, ECM-interaction, ERBb, FOXO and TGF-β signaling pathways were also found. Importantly, as demonstrated in FIG. 8, crucial pro-fibrotic factors including Fndel, Fndc3b, Col1a1 and Akt1s1 and Pxn were significantly downregulated in L-alanyl-L-glutamine treated samples, compared to untreated chronic hypoxic samples, in agreement to the in-vitro findings through Western blotting and immunofluorescence imaging.

Further analysis of the proteins found to be significantly upregulated in the L-alanyl-L-glutamine treated primary fibroblasts. Pathway analysis indicates that these proteins are associated with upregulation of GnRH signaling, Glutaminergic and axon guidance signaling. FIGS. 7A-7C summarize the findings of upregulated factors.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method of treating or preventing peripheral nerve fibrosis in a subject, the method comprising administering a therapeutically effective amount of a glutamine source to one or more peripheral nerves or one or more tissues surrounding one or more peripheral nerves, wherein the source of glutamine is administered prior to, during or after a surgical procedure involving the one or more peripheral nerves.

2. The method of claim 1, wherein one or more additional doses of the glutamine source is administered to the subject at specified periods of time after the first dose, at fixed intervals of time after the first dose, and/or just prior to closure of all surgical incisions.

3. The method of claim 1, wherein the source of glutamine is administered during a surgery involving one or more peripheral nerves.

4. The method of claim 3, wherein the surgical procedure affects the abdominal cavity of the subject.

5. The method of claim 3, wherein the surgical procedure affects the thoracic cavity of the subject.

6. The method of claim 3, wherein the surgical procedure affects the head, neck, or spine of the subject.

7. The method of claim 3, wherein the surgical procedure affects one or more limbs of the subject.

8. The method of claim 1, wherein the source of glutamine is an oligopeptide comprising glutamine.

9. The method of claim 8, wherein the oligopeptide is a dipeptide.

10. The method of claim 9, wherein the dipeptide is L-alanyl-L-glutamine.

11. The method of claim 1, wherein the source of glutamine comprises L-glutamine.

12. The method of claim 1, wherein the source of glutamine comprises D-glutamine.

13. The method of claim 1, wherein the source of glutamine is administered with an additional one or more active pharmaceutical ingredients.

14. The method of claim 1, wherein the source of glutamine is administered by injection, topical administration, transdermal administration, or by iontophoresis.

15. A method of treating or preventing adhesion in one or more peripheral nerves in a subject that is undergoing a surgical procedure, the method comprising administering a therapeutically effective amount of a glutamine source to a peripheral nerve or tissue surrounding one or more peripheral nerves, wherein the source of glutamine is administered prior to, during or after a surgical procedure involving the one or more peripheral nerves.

16. The method of claim 15, wherein the source of glutamine is an oligopeptide comprising glutamine.

17. The method of claim 16, wherein the oligopeptide is a dipeptide.

18. The method of claim 17, wherein the dipeptide is L-alanyl-L-glutamine.

19. A method of treating or preventing hypoxia-associated damage to one or more peripheral nerves of a subject, the method comprising administering to the subject during the surgical procedure a therapeutically effective amount of a glutamine source to a tissue subject to hypoxia-associated tissue damage, wherein the source of glutamine is administered prior to, during or after a surgical procedure involving the one or more peripheral nerves.

20. The method of claim 19, wherein the surgical procedure affects the abdominal cavity of the subject.

21. (canceled)

22. (canceled)

23. (canceled)

24. (canceled)

25. (canceled)

26. (canceled)

27. (canceled)

28. (canceled)

29. (canceled)

30. (canceled)

31. (canceled)
